SEQUENCE LISTING (15 sequences)

SEQ ID NO: 1 - protein, 418 aa, Mus musculus
MAFIAALGLLMARICPAVLSFPDGTLGMDAAVQEDHDNGTQLDSLTLASINTDFAFSLYKELVLKNPDTNIVFSPLSISAALALVSLGAKGNTLEEILEGLKFNLTETSEADIHQGFGHLLQRLNQPKDQVQISTGSALFIEKRQQILTEFQEKAKTLYQAEAFTADFQQPRQAKKLINDYVRKQTQGMIKELVSDLDKRTLMVLVNYIYFKAKWKVPFDPLDTFKSEFYAGKRRPVIVPMMSMEDLTTPYFRDEELSCTVVELKYTGNASALFILPDQGRMQQVEASLQPETLRKWKNSLKPRMIDELHLPKFSISTDYSLEDVLSKLGIREVFSTQADLSAITGTKDLRVSQVVHKAVLDVAETGTEAAAATGVKFVPMSAKLYPLTVYFNRPFLIMIFDTETEIAPFIAKIANPK

SEQ ID NO: 2 - DNA, 2035 bp, Mus musculus
cacgagcacagaggctggcagctggctggtttcagctctgcagactgcagaacacagaagatggctttcattgcagctctggggctcttgatggctaggatctgccctgctgtcctctccttcccagatggcacgttgggaatggatgctgcagtccaagaagaccatgacaatgggacacaactggacagtctcacattggcctccatcaacactgactttgccttcagcctctacaaggagctggttttgaagaatccagatacaaatattgtcttctccccacttagcatctcagcggccttggccctcgtgtccctgggagcaaagggcaacaccctggaagagattctagaaggtctcaagttcaatcttacagagacctctgaggcagacatccaccagggctttgggcacctcctacagaggctcaaccagccaaaggaccaggtacagatcagcacgggtagtgccctgtttattgaaaagcgccagcagatcctgacagaattccaggagaaggcaaagactctgtaccaggctgaggccttcacagcagacttccagcagcctcgacaggccaaaaagctcatcaatgactatgtgaggaaacagacccaggggatgatcaaggaactggtctcagacctggataaaaggacattgatggtgctggtgaattatatctactttaaagccaaatggaaggtgccctttgaccctcttgacacgttcaagtctgagttctacgcgggcaagaggaggcccgtgatagtgcccatgatgagcatggaggacctgaccacaccctacttccgagatgaggagctgtcctgcactgtggtggagctgaagtacacaggaaatgccagcgccctgttcatcctccctgaccagggcaggatgcagcaggtggaagccagcttacaaccagagaccctgaggaagtggaagaattctctgaaacccaggatgatagatgagctccacctgcccaagttctccatctccaccgactacagcctggaggatgtcctttcaaagctgggcatcagggaagtcttctccacacaggctgacctgtctgcaatcacaggaaccaaggatctgagagtctctcaggtggtccacaaggctgtgctggatgtggctgagacaggcacagaagcagctgctgccactggagtcaaatttgtcccaatgtctgcgaaactgtaccctctgactgtatatttcaatcggcctttcctgataatgatctttgacacagaaactgaaattgccccctttatagccaagatagccaaccccaaatgagactagaactccccaagtgttgacgcttcttcccgggagccaggcattgagcctgtctgtgggtctccatgtgcattttggcttccatgctctgcttggccttggcatgcctggattagatagtgactaactgtgttataacctcatgtacagacatccctgtgggaagtcagtgccgtgctcccagacttcttggtagcactagcccatgttcctgagcctgaaatttgtcttgtcccctacccctgctctctccctgtatctgcctccacccaaaagcctgggccccatcaagtaagctcagtcccagtttagactctggttatgtctcccttcagccttgctgtcttgatgggactgtgcaaccttacaggccaacccatatggaccaagaggaaagcttggctggcccgtgtctatccgcagcatctaggactacttggtgcccagtctgcctcactctttcatttctccaggctttgcctccaggcactgcccccctgcaggatctcctcactttgtccacatctggcacagagttagagccctcacttctgcagctgcatggggtctgtgggtcagagcagatcctttcccctggcactcctacttagaacaaagtagcctttcttttagttcccagctgaccaacctcacacaaaagaggaacaccaaccagaataaagaggtaggagcaaaggatcaataaacatgtaac

SEQ ID NO: 3 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 4 - protein, 398 aa, Homo sapiens
VSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 5 - DNA, 1352 bp, Homo sapiens
ctgggacagtgaatcgacaatgccgtcttctgtctcgtggggcatcctcctgctggcaggcctgtgctgcctggtccctgtctccctggctgaggatccccagggagatgctgcccagaagacagatacatcccaccatgatcaggatcacccaaccttcaacaagatcacccccaacctggctgagttcgccttcagcctataccgccagctggcacaccagtccaacagcaccaatatcttcttctccccagtgagcatcgctacagcctttgcaatgctctccctggggaccaaggctgacactcacgatgaaatcctggagggcctgaatttcaacctcacggagattccggaggctcagatccatgaaggcttccaggaactcctccgtaccctcaaccagccagacagccagctccagctgaccaccggcaatggcctgttcctcagcgagggcctgaagctagtggataagtttttggaggatgttaaaaagttgtaccactcagaagccttcactgtcaacttcggggacaccgaagaggccaagaaacagatcaacgattacgtggagaagggtactcaagggaaaattgtggatttggtcaaggagcttgacagagacacagtttttgctctggtgaattacatcttctttaaaggcaaatgggagagaccctttgaagtcaaggacaccgaggaagaggacttccacgtggaccaggtgaccaccgtgaaggtgcctatgatgaagcgtttaggcatgtttaacatccagcactgtaagaagctgtccagctgggtgctgctgatgaaatacctgggcaatgccaccgccatcttcttcctgcctgatgaggggaaactacagcacctggaaaatgaactcacccacgatatcatcaccaagttcctggaaaatgaagacagaaggtctgccagcttacatttacccaaactgtccattactggaacctatgatctgaagagcgtcctgggtcaactgggcatcactaaggtcttcagcaatggggctgacctctccggggtcacagaggaggcacccctgaagctctccaaggccgtgcataaggctgtgctgaccatcgacgagaaagggactgaagctgctggggccatgtttttagaggccatacccatgtctatcccccccgaggtcaagttcaacaaaccctttgtcttcttaatgattgaacaaaataccaagtctcccctcttcatgggaaaagtggtgaatcccacccaaaaataactgcctctcgctcctcaacccctcccctccatccctggccccctccctggatgacattaaagaagggttgagctgg

SEQ ID NO: 6 - protein, 423 aa, Homo sapiens [sequence omitted]

SEQ ID NO: 7 - protein, 400 aa, Homo sapiens
HPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 8 - DNA, 1522 bp, Homo sapiens
cagagttgagaatggagagaatgttacctctcctgactctggggctcttggcggctgggttctgccctgctgtcctctgccaccctaacagcccacttgacgaggagaatctgacccaggagaaccaagaccgagggacacacgtggacctcggattagcctccgccaacgtggacttcgctttcagcctgtacaagcagttagtcctgaaggcccctgataagaatgtcatcttctccccactgagcatctccaccgccttggccttcctgtctctgggggcccataataccaccctgacagagattctcaaaggcctcaagttcaacctcacggagacttctgaggcagaaattcaccagagcttccagcacctcctgcgcaccctcaatcagtccagcgatgagctgcagctgagtatgggaaatgccatgtttgtcaaagagcaactcagtctgctggacaggttcacggaggatgccaagaggctgtatggctccgaggcctttgccactgactttcaggactcagctgcagctaagaagctcatcaacgactacgtgaagaatggaactagggggaaaatcacagatctgatcaaggaccttgactcgcagacaatgatggtcctggtgaattacatcttctttaaagccaaatgggagatgccctttgacccccaagatactcatcagtcaaggttctacttgagcaagaaaaagtgggtaatggtgcccatgatgagtttgcatcacctgactataccttacttccgggacgaggagctgtcctgcaccgtggtggagctgaagtacacaggcaatgccagcgcactcttcatcctccctgatcaagacaagatggaggaagtggaagccatgctgctcccagagaccctgaagcggtggagagactctctggagttcagagagataggtgagctctacctgccaaagttttccatctcgagggactataacctgaacgacatacttctccagctgggcattgaggaagccttcaccagcaaggctgacctgtcagggatcacaggggccaggaacctagcagtctcccaggtggtccataaggctgtgcttgatgtatttgaggagggcacagaagcatctgctgccacagcagtcaaaatcaccctcctttctgcattagtggagacaaggaccattgtgcgtttcaacaggcccttcctgatgatcattgtccctacagacacccagaacatcttcttcatgagcaaagtcaccaatcccaagcaagcctagagcttgccatcaagcagtggggctctcagtaaggaacttggaatgcaagctggatgcctgggtctctgggcacagcctggcccctgtgcaccgagtggccatggcatgtgtggccctgtctgcttatccttggaaggtgacagcgattccctgtgaagctctcacacgcacaggggcccatggactcttcagtctggagggtcctggcctcctgacagcaataaataatttcgttggcc

SEQ ID NO: 9 - protein, 400 aa, Artificial Sequence
  Lex032 polypeptide; ACT/AAT hybrid
HPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKIIPMSIPVETRTIVRFNRPFLMIIVPTDTQNIFFMSKVTNPKQA

SEQ ID NO: 10 - protein, 398 aa, Homo sapiens
NSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 11 - protein, 401 aa, Artificial
  mature human ACT according to SEQ ID No. 7 having an additional N-terminal methionine
MHPNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 12 - protein, 399 aa, Artificial
  mature human ACT according SEQ ID No. 10 having an additional N-terminal Methionine
MNSPLDEENLTQENQDRGTHVDLGLASANVDFAFSLYKQLVLKAPDKNVIFSPLSISTALAFLSLGAHNTTLTEILKGLKFNLTETSEAEIHQSFQHLLRTLNQSSDELQLSMGNAMFVKEQLSLLDRFTEDAKRLYGSEAFATDFQDSAAAKKLINDYVKNGTRGKITDLIKDLDSQTMMVLVNYIFFKAKWEMPFDPQDTHQSRFYLSKKKWVMVPMMSLHHLTIPYFRDEELSCTVVELKYTGNASALFILPDQDKMEEVEAMLLPETLKRWRDSLEFREIGELYLPKFSISRDYNLNDILLQLGIEEAFTSKADLSGITGARNLAVSQVVHKAVLDVFEEGTEASAATAVKITLLSALVETRTIVRFNRPFLMIIVPTDTQNIFFMSKVTNPKQA

SEQ ID NO: 13 - protein, 399 aa, Artificial
  human AAT according Seq ID No. 4 having an additional N-terminal methionine
MVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 14 - protein, 394 aa, Homo sapiens
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 15 - protein, 395 aa, Artificial
  mature human AAT according Seq ID No. 14 having an additional N-terminal methionine
MEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

(19) United States
(12) Patent Application Publication
(10) Pub. No.: US 2006/0246074 A1
(43) Pub. Date: Nov. 2, 2006

(54) Use of alpha 1-antichymotrypsin polypeptides, or nucleic acids encoding them, in combination with alpha 1- antitrypsin polypeptides, or nucleic acids encoding them, for treatment and/or prevention of diabetes-associated and/or arterial poorly healing wounds

(76) Inventors: Jörn-Peter Halle, Penzberg (DE); Andreas Goppelt, Munich (DE)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/532,955

(22) PCT Filed: Oct. 24, 2003
(86) PCT No.: PCT/EP03/11856

§ 371 Date: Sep. 14, 2005

(30) Foreign Application Priority Data
Oct. 30, 2002 (EP) 02024200.4

(51) Int. Cl. A61K 39/395; A61K 38/54; A61K 48/00

(52) U.S. Cl. 424/146.1; 424/94.2; 514/44

(57) ABSTRACT

The invention relates to a use of alpha 1-antichymotrypsin (ACT) polypeptides, functional variants thereof and/or nucleic acids encoding them, or of a cell which is expressing an ACT polypeptide or a nucleic acid encoding it in combination with alpha-1-antitrypsin (AAT) polypeptides, functional variants thereof or nucleic acids encoding them, or of a cell which is expressing an AAT polypeptide, or a nucleic acid encoding it.

The invention relates to a use of alpha 1-antichymotrypsin (ACT) polypeptides, functional variants thereof and/or nucleic acids encoding them, or of a cell which is expressing an ACT polypeptide or a nucleic acid encoding it in combination with alpha-1-antitrypsin (AAT) polypeptides, functional variants thereof or nucleic acids encoding them, or of a cell which is expressing an AAT polypeptide, or a nucleic acid encoding it.

Skin wounds in healthy patients normally heal without any complications. However, a large number of temporal and spatial changes in the cell composition of the skin is required in order to achieve complete healing of the tissue. This process can last up to 2 years and is always associated with scar formation in non-fetal tissue. This points to the enormous complexity of the wound healing process in the skin. During the wound healing process, it is possible to distinguish different temporal and partially overlapping phases: coagulation, inflammation, proliferation and remodeling (The Physiology of Wound Healing, 1998, Oxford Institute for Continuing Education). During coagulation, blood platelets aggregate and release growth and coagulation factors. A fibrin matrix is formed, thus enabling cells to migrate into the wound. Approximately 5-7 days after injury, an inflammatory reaction is triggered by the migration of a variety of cell types into the wound, in particular neutrophilic granulocytes and monocytes which release the mediators of the inflammatory reaction. During the proliferation phase blood vessels are restored, damaged tissue is regenerated, and the regenerated tissue is remodeled. The processes during the proliferation phase comprise, in particular, neovascularization, fibroblast proliferation and reepithelialization due to the proliferation and differentiation of keratinocytes. The fibroblasts secrete several growth factors, such as PDGF and TGF-beta, which in turn regulate the synthesis and deposition of components of the extracellular matrix (ECM), such as fibronectin, laminin, glycosaminoglycans and collagen.

During the reorganization of the tissue, the ECM components, particularly collagen, are rearranged. As a result of collagen being continuously degraded and newly synthesized, the reepithelialized wound can mature, and a flat scar is formed within 2 years. Again, a large number of growth factors and chemoattractants are required for reconstructing the tissue in a coordinated manner. Thus, interleukin 1, TNF-beta and interferon-gamma influence the secretion of the ECM components. TGF-beta, PDGF and FGF are also essential for remodeling.

However, in addition to the processes which contribute to the reconstruction of destroyed structures, proteolytic processes are also important for wound healing. Proteolytic processes participate in the removal of cell debris and the degradation of intermediate structures, such as fibrin matrix. Thus, a large number of proteases are active at the edge of the wound (Martin et al., 1997, Science, 276: 75-81). For example, plasminogen is activated by plasminogen activator and the urokinase-type plasminogen activator is upregulated in migrating keratinocytes, enabling them to degrade the fibrin matrix located ahead of them. This is consistent with the observation that plasminogen-knock-out mice exhibit virtually no reepithelialization. Metalloproteinases (MMP) also play an important role. MMP9 (gelatinase B), MMP1 (interstitial collagenase) and MMP10 (stromelysin-2) are activated at various time points and are characterized by different substrate specificities. Neutrophilic granulocytes and monocytes (macrophages) also secrete proteases (Dome et al., 1999, Wound Rep. Reg. 7: 433-441; Shapiro et al., J. Rheumatol. Suppl., 1991, 27; 95-98). Monocytes contain the intracellular serine proteases elastase and cathepsin G and secrete small amounts of metalloproteinases. On the other hand, in differentiating mononuclear phagocytes, the expression of cathepsin G is suppressed and the expression of collagenase is delayed. It has been observed that, in mature macrophages, the expression of metalloproteinases is powerfully induced following stimulation (Shapiro et al., J. Rheumatol. Suppl., 1991, 27: 95-98).

With regard to chronic wounds, it is the matrix metalloproteinases which have become the focus of particular interest. Thus, the amounts of collagenolytic activity which are found in the exudates of chronic wounds are significantly increased compared to those found in the exudates of surgical wounds or open skin wounds (Yager et al., 1996, J. Invest. Dermatol. 107: 743-748). For example, a variety of investigations provide evidence that the amount of MMP-1 is markedly increased in chronic wounds and that MMP-1 is the predominant collagenase in the exudates of chronic wounds (Vaalamo et al., 1997, J. Invest. Dermatol., 109; 96-101; Nwomeh et al., 1999. J. Surg. Res., 81; 189-195). MMP8 is also expressed more strongly in chronic wounds (Yager et al., 1996, J. Invest. Dermatol. 107: 743-748; Nwomeh et al., 1999, J. Surg. Res., 81: 189-195). In addition to the MMPs belonging to the interstitial collagenase class, other MMPs, namely the gelatinases MMP2 and MMP9 and the stromelysins MMP3, MMP 10 and MMP11, have been demonstrated to be present in increased amounts in chronic wounds (Nagase and Woessner, 1999, J. Biol. Chem., 274; 21491-21494). It has been postulated that serine protease activity, namely elastase activity, is present in chronic wounds, with this activity degrading fibrin and fibronectin (Palolahti et al., 1993, Exp. Dermatol., 2: 29-37; Rao et al., 1995, J. Invest. Dermatol., 105; 572-578; Grinnell and Zhu, 1996, J. Invest. Dermatol., 106: 335-341; Herrick et al., 1997, Lab. Invest. 77: 281-288).

Another serine protease, cathepsin G, has been detected in the granulation tissue in chronic decubitus ulcers (Rogers et al., 1995; Wound Rep. Reg., 3: 273-283). In contrast, it was not possible to observe any increased amounts of cathepsin G in venous foot ulcers (Weckroth et al., 1996, J. Invest. Dermatol., 106: 1119-1124). These apparently contradicting results indicate that the term “chronic skin wounds” encompasses completely different diseases with differing pathogenetic backgrounds. In general, diabetic ulcers, venous ulcers, arterial ulcers and decubitus ulcers are discriminated. Decubitus ulcers are very deep wounds which are accompanied by necrosis, infection and maceration of the tissue. They are formed due to prolonged pressure to a given skin area. By contrast, venous ulcers are rather superficial and are caused by venous stasis whereas arterial ulcers are frequently caused by arterial occlusion diseases. Diabetic ulcers, in turn, are ulcers which often arise in diabetic patients. Among a large number of diabetes-associated complications, the late complications of diabetes also comprise characteristic changes in the skin such as frequent infections, trophic disturbances and necrobiosis lipoidica. These changes can develop into poorly healing ulcers, frequently as the result of microangiopathic disturbances. The epidemiological importance of these diseases is clear when the following survey findings are considered: 25% of patients suffering from type II diabetes frequently develop chronic ulcers (“diabetic foot”) with about half of them requiring elaborate in-patient treatment. Nevertheless these ulcers heal poorly in the end. Diabetic foot causes more hospitalization than does any other complication associated with diabetes. The number of these cases associated with diabetes type I and II is on the increase and represents approx. 2.5% of all hospital admissions.

It has been postulated that a disturbed, excessively powerful proteolytic activity is responsible for the poor healing in chronic wounds (Yager et al., 1999, Wound Rep. Reg., 7: 433-441). In normally healing wounds, an equilibrium between proteolytic and antiproteolytic activity is brought about by a broad range of protease inhibitors. This theory is supported by the observation of reduced amounts of protease inhibitors within chronic wounds such as the metalloproteinase inhibitor TIMP-1, the nonspecific protease inhibitor alpha2-macroglobulin and the elastase inhibitor alpha1-protease inhibitor (Yager et al., 1997, Wound Rep. Reg., 5:23-32; Grinnell et al., J. Invest. Dermatol., 110; 771-776; Bullen et al., J. Invest. Dermatol., 104; 236-240; Rao et al., 1995; J. Invest. Dermatol., 105: 572-578; Grinnell and Zhu, 1996, J. Invest. Dermatol., 106: 335-341). It is thought that this deficiency could lead to an incomplete “antiprotease shield” with respect to those protease inhibitors. As a consequence, the turnover rate of collagen is faster than its synthesis. These results are confirmed by other studies (Nwomeh et al., 1999, J. Invest. Dermatol., 81: 189-195; Vaalamo et al., 1996, Br. J. Dermatol., 135; 52-59; Witte et al., 1998, Surgery 124: 464-470). It has therefore been suggested to increase the antiprotease shield by raising or inducing the expression of the protease inhibitors which are reduced or lacking, respectively, by administering exogenous protease inhibitors. A large number of inhibitors of the metalloproteinases have been developed on the basis of this hypothesis (e.g. WO200073295; U.S. Pat. No. 6,166,084; WO200105397; WO200063165; WO200046189; WO200044723; DE19851184; U.S. Pat. No. 6,071,903; EP-1004578; EP-949246; WO9858925; EP-878467); none of which has, however, been authorized as a pharmaceutical which is intended for treatment chronic skin wounds and which intervenes in the protease-antiprotease equilibrium. Furthermore, research to date did not discriminate between the various diseases which are grouped under the term “chronic ulcers”. For this reason, the results of the studies cannot readily be transferred to other disturbances within the wound healing process. Thus up until now, there have been no effective therapies for chronic wound healing disturbances. Established forms of therapy are restricted to physical support of the wound healing (e.g. dressings, compresses and gels), to the scraping out of necrotic tissue and to the transplantation of skin tissues, cultured skin cells and/or matrix proteins. In recent years, growth factors have been tested for their ability to improve wound healing without, however, being able to improve the conventional therapy in a decisive manner.

In view of the strongly increasing number of diabetes II patients world-wide, and the large number of patients who are suffering from arterial ulcers, there is a large demand for novel active compounds which crucially improve the healing of poorly healing diabetes-associated and poorly healing arterial wounds. Therefore, the object of the present invention was to find a novel active and stable therapeutical composition which crucially improves the healing of poorly healing diabetes-associated and poorly healing arterial wounds.

It was found that the activity of the ACT polypeptides is selectively decreased in poorly-healing diabetic wounds compared to the observed increase in activity of ACT in normally healing wounds as well as in venous ulcers. Also, ACT was found to be specifically suitable for treating diabetic organism compared to non-diabetic organisms. However, the environment of the wound is extremely aggressive for polypeptides; i.e. ACT could be inactivated especially by elastase which is present in wounds in high amounts (see Example 2). In order to protect ACT against proteolytic inactivation and/or degradation by elastase, an ACT polypeptid and/or nucleic acids encoding this or cells expressing an ACT polypeptide or nucleic acids encoding this in combination with an AAT polypeptide and/or nucleic acids encoding this or cells expressing an AAT polypeptide or nucleic acids encoding this can be used for the treatment and/or prevention of diabetes-associated and/or arterial poorly healing wounds in mammals. In one preferred embodiment, the combination is achieved by a single ACT/AAT hybrid polypeptide, wherein the polypeptide has both elastase and chymotrypsin inhibitory function; especially preferred is a Lex032 polypeptide according to SEQ ID No. 9. Such hybrid polypeptides are e.g. known from WO95/27055 and comprise ACT polypeptides wherein the amino acid at position 358 of the mature protein is mutated to methionine, isoleucine, valine, alanine, aspartic acid, threonine and glutamic acid. Also disclosed are such polypeptides wherein the amino acids 356 through 361 of mature human ACT are substituted with Ile-Pro-XXX-Ser-Ile-Pro, wherein XXX is selected from the group of methionine, tryptophan, alanine, asparagine, aspartic acid, cysteine, glutamine, glutamic acid, glycine, histidine, isoleucine, lysine, phenylalanine, proline, serine, threonine, tyrosine and valine. Especially the polypeptide Lex032 wherein the amino acids 356 through 361 of mature human ACT are substituted with Ile-Pro-Met-Ser-Ile-Pro was found to be effective in inhibiting both elastase and chymotrypsin.

Experiment 1 which was carried out within the context of this invention demonstrates the specific efficacy of ACT for treatment of poorly healing wounds of diabetic mammals and poorly healing arterial wounds compared to treatment of healthy animals. Experiment 2 indicates that, although ACT polypeptides are abundant in chronic wound fluids of diabetic patients, the proportion of active ACT is extremely low, compared to wound fluids from acute wounds or chronic venous wounds. These findings surprisingly support the notion, that ACT in combination with AAT is especially suitable for treating and preventing diabetes-associated and/or arterial badly healing wounds. In a preferred embodiment, the diabetes-associated wounds are chronic diabetic wounds. In an especially preferred embodiment, the diabetes-associated wound is a diabetic ulcer. In a particularly preferred embodiment, the wounds are a diabetic ulcer or an arterial ulcer, most preferably a diabetic ulcer.

ACT is the major known endogenous protease inhibitor of cathepsin G. In humans, one ACT gene is known, with polymorphism having been described, particularly in the signal peptide sequence (Rubin, 1989, database entry). The sequence of the human ACT polypeptide sequence with signal peptide is shown in SEQ ID No. 6. The sequence of a mature human ACT polypeptide sequence without signal peptide is shown in SEQ ID No. 7 (Lindmark et al., Biochim. Biophys. Acta 997:90-95(1989). The sequence of another, in vivo predominant, mature ACT polypeptide is shown in SEQ ID No. 10. Both mature ACT polypeptides are obtained by cleavage of the signal peptide. ACT polypeptides which exhibit ACT activity comparable to ACT polypeptides according to SEQ ID No. 7 and 10 are polypeptides according to SEQ ID No. 11 and SEQ ID No. 12 which can be e.g. obtained by recombinant expression in yeast and which are characterized by an N-terminal Methionine followed by the sequence of the mature ACT polypeptides according to SEQ ID No. 7 and SEQ ID No. 10. Such N-terminal extensions, for example a Methionine extension which may be introduced in order to facilitate expression and/or purification do not affect ACT activity. The sequence of the human ACT nucleic acid coding sequence, coding for SEQ ID NO: 6, is shown in SEQ ID No. 8. In the case of rodents, there is evidence for a large number of genes which are homologous to ACT, with the mouse serine protease inhibitor 2-2 (spi 2-2) (SEQ ID No. 1) being the functional homologue to the human ACT gene since extensive concordance exists with regard to the reactive center, the tissue distribution and the inducibility following inflammation (Inglis et al., 1991, Gene 106: 213-220). The coding sequence of spi2-2 is shown in SEQ ID No.: 2. Other members of the rodent serine protease inhibitor 2-2 family are spi2-2 and spi3 in the rat and an spi2-2 homologue from Adodemus sylvaticus (Inglis et al., see above).

Alpha-1-antitrypsin (AAT) is the major natural inhibitor of neutrophil elastase and thereby protects ACT from proteolytic degradation and inactivation by elastase. Various alleles of AAT exist, with the most frequently occurring allele shown in SEQ ID No.: 3. The sequence of a mature polypeptide lacking part of the signal peptide is shown in SEQ ID No.: 4. The sequence of the mature AAT polypeptide is shown in SEQ ID No. 14. ACT polypeptides which exhibit AAT activity comparable to AAT polypeptides according to SEQ ID No. 4 and 14 are polypeptides according to SEQ ID No. 13 and SEQ ID No. 15 which can be e.g. obtained by recombinant expression in yeast and which are characterized by an N-terminal Methionine followed by the sequence of the mature AAT polypeptides according to SEQ ID No. 4 and SEQ ID No. 14. Such N-terminal extensions, for example a Methionine extension which may be introduced in order to facilitate expression and/or purification do not affect AAT activity. The coding sequence for the AAT polypeptide according to SEQ ID NO: 3 is shown in SEQ ID No.: 5. The major physiological function of AAT is the protection of the lower respiratory tract against destruction by human leukocyte elastase. A hereditary deficiency of AAT is associated with a 20-30 fold increased risk of developing chronic obstructive disease. In skin wounds, its function as inhibitor of human neutrophil elastase is prominent. The role of AAT in wound healing has been described in the literature (e.g. Rao et al., 1995, J. Invest. Dermatol., 105: 572-578); however the specific suitability of ACT in combination with AAT for treating diabetes-associated and/or arterial wounds has not been suggested yet.

ACT was also shown to inhibit chymases in mast cells, for which reason ACT has been described in the context with allergic reactions (Lindmark and Wallengren, Allergy, 1992 47:456-458). In addition, the role of the chymase and its inhibitor ACT has been investigated in psoriatic lesions. However, the results point to the chymase only playing a subordinate role in the pathomechanism of psoriasis (Harvima et al., 1999, Acta Derm. Veneorol., 79: 98-104) and the level of chymotrypsin inhibitor activity in the uninjured skin of psoriasis patients is unaltered (Glinski et al., 1991, Arch. Dermatol. Res., 283: 224-229). According to EP0432117, ACT could be used for treatment “skin inflammations”, “burns”, and “dermatological conditions”. However, EP0432117 does not explain the meaning, of “dermatological condition”. “Skin inflammation” refers to inflammatory diseases of a skin such as psoriasis or dermatitis, which is different from mechanically damaged skin, i.e. wounds, or from deterioration in the process of the restoration of the missing tissue in the course of wound healing. “Skin inflammation” as used in EP0432117 therefore represents a pathological condition of the skin that does not encompass poorly healing arterial wounds or poorly healing diabetes-associated wounds according to the invention. By the same token the term “dermatological condition” as used in EP0432117 does not include wounds according to the invention: the term “dermatological condition” refers to conditions of the disordered skin such as blisters, cysts, macules which represent symptoms of a disorder of the skin resulting from essentially internal processes. Wounds on the other hand are caused by mechanical forces coming from outside the body.

Lex032 (SEQ ID No.: 9) acts, as a combination of ACT and AAT in a single polypeptide chain, on both cathepsin G and elastase and is used in the treatment of pancreatitis (von Dobschuetz et al., 1999, J. Pharmacol. Exp. Ther., 290: 782-8). Lex032, like the other bifunctional mutants possessing elastase-inhibiting and cathepsin G-inhibiting properties described above have been disclosed for treatment of inflammatory diseases, for example chronic wounds and psoriasis (WO 95/27055). However, the specific suitability for treating and/or preventing diabetes-associated wounds has not been described or anticipated in the prior art until now.

Taken together, although the enzymes ACT and AAT have been known for a long time in biotechnology, and have been investigated extensively from the medical point of view, the specific suitability of ACT in combination with AAT for treating and/or preventing diabetes-associated and arterial wounds has not been described yet. Also, the combination of ACT with AAT wherein a single polypeptide is used which possesses both elastase and chymotrypsin inhibitory function (e.g. Lex032) has not been described yet for for treating and/or preventing diabetes-associated and arterial wounds. This invention shows for the first time that among the large number of different possible wounds two poorly healing wounds, namely diabetes-associated and arterial ulcers, have unexpectedly the best chances for a successful therapy by ACT in combination with AAT which would not have been considered by a skilled person.

The object of the present invention is solved by the use of a alpha 1-antichymotrypsin (ACT) polypeptide, a functional variant thereof and/or a nucleic acid encoding it, or of a cell which is expressing an ACT polypeptide or a nucleic acid encoding it, in combination with an alpha-1-antitrypsin (AAT) polypeptide, functional variant thereof or nucleic acid encoding it, or of a cell which is expressing an AAT polypeptide, or a nucleic acid encoding it, for the treatment and/or prevention of poorly healing diabetes-associated and/or poorly healing arterial wounds.

Im particular, the invention relates to the use of an ACT polypeptide having a sequence selected from the group comprising SEQ ID No. 1, SEQ ID No. 6, SEQ ID No.: 7, SEQ ID No. 10, SEQ ID No. 11 and SEQ ID No. 12, or a functional variant thereof or of a nucleic acid encoding it, in combination with an AAT polypeptide having a sequence selected from the group comprising SEQ ID No. 3, SEQ ID No. 4, SEQ ID No. 13, SEQ ID No. 14 and SEQ ID No. 15, or a functional variant thereof or of a nucleic acid encoding it, for treatment and/or prevention of diseases which are selected from poorly healing diabetes-associated wounds and/or poorly healing arterial wounds.

In a preferred embodiment, the invention relates to the use of ACT polypeptide having a sequence selected from the group comprising SEQ ID No.: 7, SEQ ID No. 10, SEQ ID No. 11 and SEQ ID No. 12 in combination with an AAT having a sequence selected from the group comprising SEQ ID No.: 4, SEQ ID No. 13, SEQ ID No. 14 and SEQ ID No. 15, for treatment and/or prevention of diseases which are selected from poorly healing diabetes-associated wounds and/or poorly healing arterial wounds.

In another preferred embodiment, the invention relates to the use of a polypeptide according to SEQ ID No.: 9 or a functional variant thereof with one or two of the first two amino acids (His-Pro) missing and/or an N-terminal extension, preferably an N-terminal Methionine extension for treatment and/or prevention of diseases which are selected from poorly healing diabetes-associated wounds and/or poorly healing arterial wounds.

The term “functional variants” is to be understood as meaning variants of the ACT or AAT polypeptides, respectively, which can be used in accordance with the invention, which variants possess protease inhibitor specificity with regard to capthepsin G or neutrophil elastase which is similar to that of the native ACT polypeptide and AAT polypeptide, respectively. “Functional variants” also comprise ACT or AAT variants which, on a single polypeptide chain, possess both elastase and cathepsin G inhibitory action which is similar to that both of the native ACT polypeptide and AAT polypeptide; such functional variants and nucleic acids encoding these are combinations of ACT and AAT according to the invention. For example, functional variants of ACT or AAT polypeptides possess at least approximately 70%, in particular at least approx. 80%, especially at least approx. 90%, more preferred at least 95%, even more preferred at least 98% sequence identity with one of the sequences SEQ ID No. 1, SEQ ID No.: 3, SEQ ID No.: 4, SEQ ID No.: 6, SEQ ID No.: 7, SEQ ID No.: 9, SEQ ID No. 10, SEQ ID No. 11, SEQ ID No. 12, SEQ ID No. 13, SEQ ID No. 14 or SEQ ID No. 15 and have the protease inhibitory activity described above. Functional variants of the polypeptide can also be parts of the polypeptides used in accordance with the invention which, when compared with the native ACT or AAT polypeptides, do exhibit similar protease inhibitor activity, respectively. For example, the first amino acid, i.e. methionine, can be missing in a polypeptide without there being any significant change in the function of the polypeptide. Also, as mentioned above, an N-terminal Methionine may be added to an ACT or AAT polypeptide or functional variants thereof, e.g. a methionine may be added to the N-terminus of a mature ACT or AAT polypeptide without there being any significant change in the function of the polypeptide. N— and/or C-terminal and/or internal deletions of the polypeptide in the range of approx. 1-60, preferably of approx. 1-30, in particular of approx. 1-15, especially of approx. 1-5, amino acids are also included provided the protease inhibitor specificity remains essentially unaltered as compared with that of the respective native polypeptide. Particular preference is given to deletions which affect the signal peptide, or parts thereof, at the N terminus of an ACT or AAT polypeptide; e.g. it was shown that an ACT polypeptide having the last 4 amino acids of the signal peptides followed by the sequence of the mature ACT polypeptide according to SEQ ID No. 10 retains full ACT activity (U.S. Pat. No. 5,367,064). Especially preferred are ACT polypeptides which are characterized in that all or part of the signal peptide of an ACT polypeptide according to SEQ ID No. 6 is missing, especially ACT polypeptides according to SEQ ID No. 7 and SEQ ID No. 10. ACT polypeptides having an additional N-terminal Methionine are also preferred ACT polypeptides according to the invention, especially ACT polypeptides according to SEQ ID No. 11 and SEQ ID No. 12. Especially preferred are AAT polypeptides which are characterized in that all or part of the signal peptide of an AAT polypeptide according to SEQ ID No. 3 is missing, especially AAT polypeptides according to SEQ ID No. 4 and SEQ ID No. 14. AAT polypeptides having an additional N-terminal Methionine added are also preferred AAT polypeptides according to the invention, especially AAT polypeptides according to SEQ ID No. 13 and SEQ ID No. 15.

Other examples of such variants, whose protease inhibitor specificity is not significantly altered as compared with that of the native polypeptide, are the polypeptides which are homologous with the polypeptides used in accordance with the invention and which are derived, in particular, from organisms other than humans or mice, preferably from non-human mammals such as monkeys, pigs and rats. Other examples of polypeptides which are encoded by different alleles of the gene, in different individuals or in different organs, and which, when compared with the native polypeptide, do not exhibit any protease inhibitor specificity which is significantly altered as compared with that of the native polypeptide in an organism. It is known for example that several AAT alleles exist. Furthermore, a posttranslational or cotranslational modification of the polypeptide chain which is present in the native state can be missing or be altered. In particular, covalently bound sugar residues can be missing or be altered, and activity still being similar. For example, human ACT can be produced in E. coli in an unglycosylated form and the specific enzymatic activity is still retained (e.g. U.S. Pat. No. 5,079,336). Similarly, ACT and AAT and their functional variants may be produced in bacteria or yeast strains to obtain unglycosylated functional variants. In this case, recombinant production of the polypeptides useable according to the invention may be effected by expressing the coding sequence for the mature forms plus an N-terminal Methionine for initiating expression. Such polypeptides having an N-terminal methionine are preferred ACT and AAT polypeptides according to the invention.

Preferred functional variants are ACT and AAT polypeptides in which the signal peptide, or a part thereof, is deleted, for example a combination of polypeptides according to SEQ ID No. 4 or SEQ ID No. 14 together with SEQ ID No. 7 or SEQ ID No. 10 is especially preferred for use according to the invention. Furthermore, preferred functional variants are ACT and AAT polypeptides which have N-terminal extensions, preferably a Methionine extension, even more preferred have an N-terminal extension to an ACT or AAT polypeptide in which the signal peptide, or a part thereof, is deleted. Such functional variants of ACT and AAT polypeptides may be combined; e.g. an ACT polypeptide having all of the signal peptide deleted, for example ACT polypeptide according to SEQ ID No. 10 can be combined with a AAT polypeptide in which all of the signal sequence was deleted and an N-terminal Methionine was then added, for exaple AAT polypeptide according to SEQ ID No. 15. A combination of a polypeptide selected from the group comprising polypeptides according to SEQ ID No. 4, SEQ ID No. 13, SEQ ID No. 14 and SEQ ID No. 15 together with a polypeptide selected from the group comprising polypeptides according to SEQ ID No. 7, SEQ ID No. 10, SEQ ID No. 11 and SEQ ID No. 12 is especially preferred for use according to the invention.

Analogously, preferred polypeptides according to the invention are functional variants of polypeptide Lex032 according to SEQ ID No. 9, in which one or two of the first two amino acids (i.e. His-Pro) of SEQ ID No. 9 are missing and/or to which an N-terminal Methionine was added. Such polypeptides are preferred polypeptides useable according to the invention.

Furthermore, the ACT and AAT which can be used in accordance with the invention can be glycosylated, partially glycosylated or unglycosylated. In a preferred embodiment, the ACT and AAT polypeptides are unglycosylated. Such ACT and AAT polypeptides can be obtained by recombinant expression e.g. in bacteria or in yeast strains. In another preferred embodiment, the ACT and AAT polypeptides are glycosylated. Such ACT and AAT polypeptides can be obtained by isolation from mammalian tissue or by recombinant expression in mammalian cells.

A “signal peptide” according to the invention is to be understood as the N-terminal part of an ACT or AAT polypeptide, which is necessary for export out of a mammalian cell during expression and which is eventually removed during export out of a mammalian cell, thereby liberating the mature forms of the proteins. In case of ACT, two different signal peptides exist, comprising amino acids 1-23 and 1-25 of the ACT polypeptide according to SEQ ID No. 6; the corresponding mature ACT polypeptides are polypeptides according to SEQ ID No. 7 and SEQ ID No. 10. In case of AAT, one signal peptide was identified, comprising amino acids 1-24 of the AAT polypeptide according to SEQ ID No. 3; the corresponding mature AAT polypeptide is polypeptide according to SEQ ID No.14.

The term “coding nucleic acid” or “nucleic acid encoding . . . ” (an ACT or AAT polypeptide) relates to an RNA or DNA sequence which encodes an ACT or AAT polypeptide which can be used in accordance with the invention or a functional variant thereof or a precursor stage thereof, for example a propolypeptide or a prepropolypeptide. The polypeptide can be encoded by a full-length sequence or any part of the coding sequence as long as the polypeptide is a functional variant.

The term “variants” denotes all the DNA sequences which are complementary to a DNA sequence (reference sequence), which encode polypeptides used in accordance with the invention, especially polypeptides according SEQ ID No. 1, SEQ ID No.: 3, SEQ ID No. 4, SEQ ID No.: 6, SEQ ID No.:7, SEQ ID No. 9, SEQ ID No. 10, SEQ ID No. 11, SEQ ID No. 12, SEQ ID No. 13, SEQ ID No. 14 or SEQ ID No. 15 or their functional variants and which exhibit at least approx. 70%, in particular at least approx. 80%, especially at least approx. 90%, sequence identity with the reference sequence. The term “variants” furthermore denotes all the DNA sequences which are complementary to the reference sequence and which hybridize with the reference sequence under stringent conditions and encode a polypeptide which exhibits essentially the same activity as does the polypeptide encoded by the reference sequence, and also their degenerate forms. It is known that small changes can be present in the sequence of the nucleic acids which can be used in accordance with the invention; for example, without the property of a functional variant being lost, these changes can be brought about by the degeneracy of the genetic code or by nontranslated sequences which are appended at the 5′ end and/or the 3′ end of the nucleic acid. This invention therefore also encompasses so-called “variants” of the previously described nucleic acids.

The term “stringent hybridization conditions” is to be understood, in particular, as meaning those conditions in which a hybridization takes place, for example, at 60° C. in 2.5×SSC buffer, followed by several washing steps at 37° C. in a lower buffer concentration, and remains stable.

Sequence identity is understood as degree of identity (% identity) of two sequences, that in the case of polypeptides can be determined by means of for example BlastP 2.0.1 and in the case of nucleic acids by means of for example BLASTN 2.014, wherein the filter is set off and BLOSUM is 62 (Altschul et al., 1997, Nucleic Acids Res., 25:3389-3402). “Sequence homology” is understood as similarity (% positives) of two polypeptide sequences determined by means of for example BlastP 2.0.1 wherein the Filter is set off and BLOSUM is 62 (Altschul et al., 1997, Nucleic Acids Res., 25:3389-3402).

Within the meaning of the present invention, diabetes-associated wounds which heal poorly are to be understood as being skin lesions in mammals and humans suffering from diabetes. Examples of such skin lesions are to be understood as being, in particular, ulcers caused by diabetes, i.e. diabetic ulcers, for example ulcus cruris arteriosum, necrobiosis lipoidica and ulcera arteriosa in diabetic patients, and delayed wound healing which is caused by arteriosclerotic destruction of the blood vessels in diabetic patients.

Within the meaning of the invention, arterial wounds which heal poorly are to be understood, for example, as being ulcera arteriosa, i.e. arterial ulcers and retarded wound healing which is caused by arteriosclerotic destruction of the blood vessels.

Within the meaning of the present invention the function of ACT is to be understood as the activity which ACT exerts onto protease Cathepsin G. The function of ACT also encompasses the activity of ACT in the form of complexes of ACT and Cathepsin G, i.e. the Cathepsin G:ACT complex which binds to receptors, e.g. the serpin-enzyme complex receptor (SECR) (Chen et al., 1993, Neurology 43: 1223-7; Perlmutter et al., 1990 PNAS: 87: 3753-7).

Within the meaning of the present invention, activity of ACT preferably encompasses the binding and inhibition of Cathepsin G protease. Suitable assays to determine the activity of ACT are protease inhibitory assays such as, e.g. Example 2 or Heidtmann et al., 1990, Clin Chem 36: 2077-2081.

Within the meaning of the present invention the function of AAT is to be understood as the binding and inhibition of the protease neutrophil elastase. A suitable assay is described in Rubin et al., 1994, Biochemistry, 33: 7627-7633.

A “similar protease inhibitor activity” within the meaning of the invention is a protease inhibitor activity of a ACT or AAT functional variant that is preferably not less than 0.1%, more preferably not less than 1%, even more preferred not less than 50% and most preferred not less than 90% of the activity of the native enzyme.

The nucleic acids which can be used in accordance with the invention and which encode ACT or AAT polypeptides which can be used in accordance with the invention, or their functional variants, are preferably DNA or RNA, preferably a DNA, in particular a double-stranded DNA. Furthermore, in one embodiment, the sequence of the nucleic acids can be characterized by the fact that it possesses at least one intron and/or a polyA sequence.

In general, preference is given to a double-stranded DNA for expressing the relevant gene, both for preparing a polypeptide which can be used in accordance with the invention and in association with a vector which is applicable in gene therapy and can be used in accordance with the invention, with particular preference being given to the DNA region which encodes the polypeptide. In eukaryotes, this region begins with the first start codon (ATG) which is located in a Kozak sequence (Kozak, 1987, Nucleic. Acids Res. 15:8125-48) and extends to the next stop codon (TAG, TGA or TAA) which is located in the same reading frame as the ATG. In the case of prokaryotes, this region begins with the first AUG (or GUG) after a Shine-Dalgarno sequence and ends with the next stop codon (TAG, TGA or TAA) which is located in the same reading frame as the ATG.

Furthermore, it is possible to use a nucleic acid which has been prepared synthetically for implementing the invention. Thus, the nucleic acid which is used in accordance with the invention can, for example, be synthesized chemically, e.g. in accordance with the phosphotriester method, making use of the DNA sequences described in the sequence listing and/or making use of the protein sequences which are likewise described in this table by referring to the genetic code (see, e.g., Uhlmann, E. & Peyman, A. (1990) Chemical Reviews, 90, 543-584, No. 4).

In a particularly preferred embodiment of the invention, at least one nucleic acid which can be used in accordance with the invention is contained in an expression cassette in a vector, preferably in a vector which is applicable in gene therapy. The invention also comprises the use of a vector expressing a fusion protein useable according to the invention. The vector which is applicable in gene therapy preferably contains tissue-specific, wound-specific or skin-specific, cell cycle-specific, cell type-specific, metabolism-specific or constitutively active regulatory sequences which are functionally linked to the previously described nucleic acid.

Examples of suitable regulatable elements which permit constitutive expression in eukaryotes are promoters which are recognized by RNA polymerase III or viral promoters, CMV enhancer, CMV promoter (see also Example 3), SV40 promoter or LTR promoters, e.g. derived from MMTV (mouse mammary tumor virus; Lee et al. (1981) Nature 214, 228-232) and other viral promoter and activator sequences which are derived from, for example, HBV, HCV, HSV, HPV, EBV, HTLV or HIV.

Examples of regulatable elements which permit inducible expression in eukaryotes are the tetracycline operator in combination with an appropriate repressor (Gossen M. et al. (1994) Curr. Opin. Biotechnol. 5, 516-20).

The expression of nucleic acids which can be used in accordance with the invention preferably takes place under the control of tissue-specific promoters, with skin-specific promoters, such as the human K10 promoter (Bailleul et al., 1990. Cell 62: 697-708), the human K14 promoter (Vassar et al., 1989, Proc. Natl. Acad. Sci. USA 86: 1563-67) or the bovine cytokeratin IV promoter (Fuchs et al., 1988; The Biology of Wool and Hair (eds.: G. E. Rogers, et al.), pp. 287-309. Chapman and Hall, London/New York) being particularly to be preferred.

Other examples of regulatable elements which permit tissue-specific expression in eukaryotes are promoters or activator sequences from promoters or enhancers of those genes which encode proteins which are only expressed in particular cell types.

Examples of regulatable elements which permit cell cycle-specific expression in eukaryotes are promoters of the following genes: cdc25A, cyclin A, cyclin E, cdc2, E2F, B-myb and DHFR (Zwicker J. and Müller R. (1997) Trends Genet. 13, 3-6).

Examples of regulatable elements which permit metabolism-specific expression in eukaryotes are promoters which are regulated by hypoxia, by glucose deficiency, by phosphate concentration or by heat shock.

An example of a regulatable element which permits keratinocyte-specific expression in skin is the FiRE element (Jaakkola et al., 2000, Gen. Ther., 7: 1640-1647). The FiRE element is an AP-1-driven, FGF-inducible response element of the syndecan-1 gene (Jaakkola et al., 1998, FASEB J., 12: 959-9).

In order to enable the nucleic acids which can be used in accordance with the invention to be introduced into a eukaryotic or prokaryotic cell by means of transfection, transformation or infection, and thereby enabling the polypeptide to be expressed, the nucleic acid can be present as a plasmid, or as a part of a viral or non-viral vector. Particularly suitable viral vectors in this context are: baculoviruses, vaccinia viruses, adenoviruses, adeno-associated viruses and herpesviruses. Particularly suitable non-viral vectors in this context are: liposomes, virosomes, cationic lipids and polylysine-conjugated DNA.

Examples of vectors which are applicable in gene therapy are viral vectors, for example adenoviral vectors or retroviral vectors (Lindemann et al., 1997, Mol. Med. 3: 466-76; Springer et al., 1998, Mol. Cell. 2: 549-58). Eukaryotic expression vectors are suitable for use in gene therapy when present in isolated form since naked DNA can penetrate into skin cells when applied topically (Hengge et al., 1996, J. Clin. Invest. 97: 2911-6; Yu et al., 1999, J. Invest. Dermatol. 112: 370-5).

Vectors which are applicable in gene therapy can also be obtained by complexing the nucleic acid which can be used in accordance with the invention with liposomes, since this makes it possible to achieve a very high efficiency of transfection, particularly of skin cells (Alexander and Akhurst, 1995, Hum. Mol. Genet. 4: 2279-85). In lipofection, small, unilamellar vesicles consisting of cationic lipids are prepared by subjecting the liposome suspension to ultrasonication. The DNA is bound ionically on the surface of the liposomes, specifically in a relationship which is such that a positive net charge remains and 100% of the plasmid DNA is complexed by the liposomes. In addition to the DOTMA (1,2-dioleoyloxypropyl-3-trimethylammonium bromide) and DPOE (dioleoylphosphatidylethanolamine) lipid mixtures employed by Felgner et al. (1987, see above), a large number of new lipid formulations have by now been synthesized and tested for their efficiency in the transfection of various cell lines (Behr, J. P. et al. (1989), Proc. Natl. Acad. Sci. USA 86, 6982-6986; Felgner J. H. et al. (1994) J. Biol. Chem. 269, 2550-2561; Gao, X. and Huang L. (1991), Biochim. Biophys. Acta 1189, 195-203). Examples of the new lipid formulations are DOTAP N-[1-(2,3-dioleoyloxy)propyl]-N,N,N-trimethylammonium ethyl sulfate or DOGS (TRANSFECTAM; dioctadecylamidoglycylspermine). The Cytofectin GS 2888 cationic lipids have also proved to be very well suited for transfecting keratinocytes in vitro and in vivo (U.S. Pat. No. 5,777,153; Lewis et al., 1996, Proc. Natl. Acad. Sci. USA, 93: 3176-3181). Auxiliary substances which increase the transfer of nucleic acids into the cell can, for example, be proteins or peptides which are bound to DNA or synthetic peptide-DNA molecules which make it possible to transport the nucleic acid into the nucleus of the cell (Schwartz et al. (1999) Gene Therapy 6, 282; Brandén et al. (1999) Nature Biotech. 17, 784). Auxiliary substances also encompass molecules which enable nucleic acids to be released into the cytoplasm of the cell (Planck et al. (1994) J. Biol. Chem. 269, 12918; Kichler et al. (1997) Bioconj. Chem. 8, 213). Liposomes are a pharmaceutically acceptable carrier within the meaning of the present invention. Liposomes comprise multilamellar vesicles (MLVs), small unilamellar vesicles (SUVs) and large unilamellar vesicles (LUVs).

Methods for preparing liposome-nucleic acid complexes are known to the skilled person (e.g. Straubinger et al., 1983, in Methods of Immunology, 101: 512-527; Szoka et al., 1978, Proc. Natl. Acad. Sci. USA, 75: 4194-4198), The term “liposomes” encompasses, for example, liposomal compositions which are disclosed in U.S. Pat. No. 5,422,120, WO 95/13796, WO 94/23697, WO 91/14445 and EP 524,968 B1. Liposomes can be used for nucleic acids usable according to the invention as well as for polypeptides usable according to the invention or for both as a pharmaceutical carrier, preferably they are used as pharmaceutical carriers for the nucleic acids according to the invention. The therapeutically active substance can also be conjugated to the liposome or it can be conjugated to a hydrogel polymer, wherein the hydrogel polymer (or a component of the hydrogel polymer) is conjugated to a liposome or can be enclosed by a liposome. Another especially suitable form of gene therapeutical vectors can be obtained by applying the nucleic acid usable according to the invention to gold particles and apply these topically with the aid of the so called “gene gun” by shooting them into the skin or cells (Example 1; Wang et al., 1999, J. Invest. Dermatol., 112: 775-81, Tuting et al., 1998, J. Invest. Dermatol., 111: 183-8). Nucleic acids encoding AAT and ACT polypeptides may then be applied together at the same time or temporally separated from each other.

The term “liposomes” encompasses, for example, liposomal compositions which are disclosed in U.S. Pat. No. 5,422,120, WO 95/13796, WO 94/23697, WO 91/14445 and EP 524,968 B1. Liposomes can be used as a pharmaceutical carrier for the nucleic acids which can be used in accordance with the invention and the polypeptides which can be used in accordance with the invention; they are preferably used as a pharmaceutical carrier for the nucleic acids which can be used in accordance with the invention. The therapeutically active substance can be conjugated to the liposome or it can be conjugated to a hydrogel polymer, with it being possible for the hydrogel polymer (or a component of the hydrogel polymer) to be conjugated to a liposome or to be enclosed by a liposome. Another particularly preferred form of vector for gene therapy can be obtained by applying the nucleic acid which is used in accordance with the invention to gold particles and using a Gene Gun to administer the particles topically by shooting the loaded particles into the skin or cells (Example 1; Wang et al., 1999, J. Invest. Dermatol., 112: 775-81, Tuting et al., 1998, J. Invest. Dermatol, 111: 183-8). Devices for performing intradermal injection using pressure have been disclosed, for example, in U.S. Pat. No. 5,630,796.

Another form of vector which is applicable in gene therapy can be prepared by introducing “naked” expression vectors into a biocompatible matrix, for example a collagen matrix. This matrix can, for example, be introduced into diabetes-associated and/or arterial wounds in order to transfect the immigrating cells with the expression vector and to express the polypeptides used in accordance with the invention in the cells (Goldstein and Banadio, U.S. Pat. No. 5,962,427).

For the use of the previously described nucleic acid in gene therapy it is also advantageous if the part of the nucleic acid which encodes the polypeptide contains one or more non-coding sequences, including intron sequences, preferably between the promoter and the start codon for the polypeptide (see Example 1) and/or a polyA sequence, in particular the naturally occurring polyA sequence or an SV40 virus polyA sequence, in particular at the 3′ end of the gene since this thereby makes it possible to stabilize the mRNA (Jackson, R. J. (1993) Cell 74, 9-14 and Palmiter, R. D. et al. (1991) Proc. Natl. Acad. Sci. USA 88, 478-482).

For production of the polypeptides, cells can be either prokaryotic or eukaryotic cells. Examples of prokaryotic cells are E. coli, and examples of eukaryotic cells are Saccharomyces cerevisiae or insect cells. Thus, E. coli cells have, for example, proved to be suitable cells for expressing human ACT (Rubin et al., 1990, J. Biol. chem., 265: 1199-1207). The use of E. coli cells for preparing ACT and AAT polypeptides which can be used in accordance with the invention constitutes a preferred embodiment. In a further preferred embodiment the ACT and AAT polypeptides are produced in yeast strains. The polypeptides which can be used in accordance with the invention are prepared, for example, by expressing the previously described nucleic acid in a suitable expression system, as already described above, using methods which are well known to the skilled person. Examples of suitable cells are the E. coli strain DHS, HB101 or BL21, the yeast strain Saccharomyces cerevisiae, the insect cell line Lepidopteran, e.g. from Spodoptera frugiperda, or the animal cells COS, Vero, 293, HaCaT and HeLa, all of which are generally available.

Another preferred embodiment of the invention is the use of an ACT polypeptide or a functional variant thereof in combination with an AAT polypeptide or a functional variant thereof which can be used in accordance with the invention in the form of a fusion protein for prevention and/or treatment of diabetes-associated and/or arterial wounds which heal poorly, which fusion protein is prepared using a previously described nucleic acid which can be used in accordance with the invention.

This involves preparing fusion proteins which contain the above-described polypeptides which can be used in accordance with the invention or their functional variants, with the fusion proteins themselves already constituting a functional variant of one of the previously described ACT or AAT polypeptides or only being a functional variant after the fusion moiety has been eliminated. These fusion proteins include, in particular, fusion proteins which have a content of approx. 1-300, preferably approx. 1-200, particularly preferably approx. 1-150, in particular approx. 1-100, and especially approx. 1-50, foreign amino acids. Examples of such peptide sequences are prokaryotic peptide sequences which can be derived, for example, from E. coli galactosidase.

Other preferred examples of peptide sequences for fusion proteins are peptides which facilitate detection of the fusion protein; they include, for example, green fluorescent protein or variants thereof.

The polypeptides which can be used in accordance with the invention can also be prepared synthetically. Thus, the entire polypeptide, or parts thereof, can, for example, be synthesized by means of classical synthesis (Merrifield technique). Particular preference is given to using polypeptides which have been prepared recombinantly using one of the previously described nucleic acids. Furthermore, ACT and AAT polypeptides can be isolated from an organism or from tissue or cells and then used in accordance with the invention. Thus, it is possible, for example, to purify polypeptides which can be used in accordance with the invention from human serum, for example (e.g. Abdullah et al., 1983, Arch. Biochem, Biophys., 225:306-312). Furthermore, it is possible to prepare cell lines from ACT-expressing cells, which cell lines can then be used for isolating ACT. Thus, active ACT can, for example, be prepared by recombinant expression in E. coli cells (Rubin et al., 1990, J. Biol. Chem. 265: 1199-1207).

It is possible to add on at least one “polypeptide tag” for the purpose of purifying the previously described proteins. For example, suitable protein tags enable the proteins which are to be purified to be absorbed with high affinity to a matrix. This is then followed, for example, by the following steps: stringent washing with suitable buffers without eluting the complex to any significant extent, and, subsequently, specific elution of the absorbed complex. Examples of the protein tags which are known to the skilled person are a (His)₆ tag, an Myc tag, a FLAG tag, a hemagglutinin tag, a glutathione transferase (GST) tag, a tag consisting of a an intein flanked by an affinity chitin-binding domain, and a maltose-binding protein (MBP) tag. These protein tags can be located N-terminally, C-terminally and/or internally.

In another embodiment, antibodies or antibody fragments are useable according to the invention wherein one antibody is a catalytic antibody which increases the activity of an ACT polypeptide and a second antibody is a catalytic antibody which increases the activity of an AAT polypeptide. Examples of catalytic antibodies are found for example in Tramontano et al., 1986, Science 234: 1566-70.

The present invention also relates to the use of an alpha 1-antichymotrypsin (ACT) polypeptide, a functional variant thereof and/or a nucleic acid encoding it, or of a cell which is expressing an ACT polypeptide or a nucleic acid encoding it, in combination with an alpha-1-antitrypsin (AAT) polypeptide, a functional variant thereof or a nucleic acid encoding it, or with a cell which is expressing an AAT polypeptide or a nucleic acid encoding it, for producing a pharmaceutical for the treatment and/or prevention of poorly healing diabetes-associated and/or poorly healing arterial wounds.

The present invention also relates to a method of manufacturing a pharmaceutical composition for treatment, and/or prevention of diseases selected from poorly healing diabetes-associated wounds and/or poorly healing arterial wounds wherein an ACT polypeptide, or a nucleic acid coding for the ACT polypeptide, or a cell expressing the ACT polypeptide or a nucleic acid coding for the ACT polypeptide, is combined with an AAT polypeptide, or a nucleic acid coding for the AAT polypeptide, or a cell expressing the AAT polypeptide or a nucleic acid coding for the AAT polypeptide.

Preferably, an ACT polypeptide having a sequence selected from the group comprising SEQ ID No.: 1, SEQ ID No.: 6, SEQ ID No.: 7, SEQ ID No. 10, SEQ ID No. 11 and SEQ ID No. 12 or a functional variant thereof or a nucleic acid encoding it, is combined with an AAT polypeptide having a sequence selected from the group comprising SEQ ID No.: 3, SEQ ID No.: 4, SEQ ID No. 13, SEQ ID No. 14 and SEQ ID No. 15 or a functional variant thereof or a nucleic acid encoding it.

In a preferred embodiment, an ACT polypeptide selected from the group of polypeptides having a sequence according to SEQ ID No.: 7, SEQ ID No. 10, SEQ ID No. 11 and SEQ ID No. 12 is combined with an AAT polypeptide selected from the group of polypeptides having a sequence according to SEQ ID No.: 4, SEQ ID No. 13, SEQ ID No. 14 and SEQ ID No. 15.

In one embodiment, a polypeptide having a sequence according to SEQ ID No.: 9 or a functional variant thereof having one or two of the two N-terminal amino acids (His and Pro) missing and/or having an N-terminal Methionine added is used, preferably a polypeptide having a sequence according to SEQ ID No.: 9.

In one embodiment, the wounds are a diabetic ulcer or an arterial ulcer, wherein preferably the wounds are a diabetic ulcers.

In one embodiment, the polypeptides are unglycosylated.

In one embodiment, the polypeptides are isolated from tissue, preferably from serum.

Preferably, the ACT and AAT polypeptides are administered separately.

In one embodiment, the nucleic acid is employed in the form of an expression vector.

Preferably, the expression vector is a vector applicable in gene therapy.

In a preferred embodiment, the cells are autologous or allogenic cells, wherein, preferably, the cells are skin cells such as keratinocytes, fibroblasts or endothelial cells.

The therapy of the diabetes-associated and/or arterial wounds which heal poorly can be effected in a conventional manner, e.g. using dressings, plasters, compresses or gels which contain the pharmaceuticals which can be used in accordance with the invention. Thus, it is possible to administer the pharmaceuticals topically and locally in order to exert an immediate and direct effect on wound healing. The topical administration of therapeutic compositions can be effected, for example, in the form of a solution, an emulsion, an cream, an ointment, a foam, an aerosol spray, a gel matrix, a sponge, drops or washings. Suitable additives or auxiliary substances are isotonic solutions, such as physiological sodium chloride solutions or sodium alginate, demineralized water, stabilizers, collagen containing substances such as Zyderm II or matrix-forming substances such as povidone. To generate a gel basis, formulations, such as aluminum hydroxide, polyacrylacid derivatives, such as Carbopol®, cellulose derivatives, such as carboxymethyl cellulose are suitable. These gels can be prepared as hydrogels on a water basis or as oleogels with low- and high molecular paraffines or vaseline and/or yellow or white wax. As emulsifier alkali soaps, metal soaps, amine soaps or partial fatty acid esters of sorbitants can be used whereas lipids can be added as vaseline, natural and synthetic waxes, fatty acids, mono-, di-, triglycerides, paraffin, natural oils, such as cocos oil, synthetic fats, such as Miglyol®. These forms of administration are preferred for using at least one ACT polypeptide which can be used in accordance with the invention. The pharmaceuticals according to the invention can also, where appropriate, be administered topically and locally, in the region of the wound, in the form of liposome complexes or gold particle complexes. This form of administration is preferred for vectors which are applicable in gene therapy and which contain a nucleic acid which can be used in accordance with the invention. Topical formulations for ACT and/or AAT polypeptides are well known in the prior art; e.g. in U.S. Pat. No. 6,294,181, U.S. Pat. No. 6,096,327; U.S. Pat. No. 5,008,242, EP 0 512 090, EP 0 432 117 and U.S. Pat. No. 5,190,917.

Furthermore, the treatment can be effected using a transdermal therapeutic system (TTS), which enables the pharmaceuticals according to the invention to be released in a temporally controlled manner. To improve the penetration of the administered drug through the membrane, additives such as ethanol, urea or propylene glycol can be added in addition to polymeric auxiliaries, such as Eudragit®. TTS have been disclosed, for example, in EP 0 944 398 A1, EP 0 916 336 A1, EP 0 889 723 A1 or EP 0 852 493 A1.

In another embodiment, the pharmaceuticals according to the invention can also be administered as a cell expressing a combination of polypeptides of the invention which is then secreted to the wound site. Preferred cells are autologous or allogenic cells, particularly preferred are skin cells such as keratinocytes, fibroblasts or endothelial cells. A suitable carrier for administering those modified cells would be a micro carrier consisting of bio compatible materials, such as, for example a dextran matrix (U.S. Pat. No. 5,980,888).

However, the treatment with the pharmaceuticals according to the invention can also be effected systemically using parenteralia such as injections or infusions or peroralia. Injections can be applied subcutaneously, intradermally, intraepithelially, intrafusally, intramuscularly, intravenous, intracutaneously, intraperitoneally, or intrathecally and can be formulated as solutions, suspensions, as concentrates or lyophilized powder which can be diluted in isotonic solutions. Infusions can also be applied as isotonic solutions or as fat emulsions, as “high tech” formulations such as liposomes, micro emulsions, nanospheres, microspheres, microcapsules. To improve the adsorption of parenteral applied drugs to proteins or polymers and/or to reduce the association of the drug with glass or plastic surfaces, substances such as albumin, organic solvents, plasmaexpander or surface active substances could be used. Suitable auxiliaries for the production of parenteral applied drugs are isotonic substances, such as sodium chloride, isohydric substances, such as sodium hydrogen carbon acid or tensides or surface active substances and emulsifiers, such as Tween®, Cremophor®, or complex inducing substances such as urea and citrate. An alternative parenteral administration would be nasal, oral cavity or rectal administration. For nasal and oral cavity applications, administrations, such as a spray, an ointment, an aerosols or drops which can be inhaled could be used. Suitable propellants would be tetrahydrofluran or heptafluorpropan but manual pressure systems would also be suitable. These propellants could contain surface active substances such as isopropylmyristat. For the rectal administration clyster preparations, such as tablets or capsules or suppositories. As auxiliary substances, Witepsol®, Massa Estarium® or Novata® could be used. A modified form of parenteral release would be the usage of depots which are implanted under the skin which consist preferably on the basis of biological degradable polymers.

A further systemically applied form is the oral administration such as tablets, fizzy tablets, capsules, pellets, dragees, powder, granules, pastilles, chewing gum, drops or suspensions. Suitable auxiliaries are for example starch, lactose, talcum, stearine acid, cellulose, PVP or Aerosil®. Suitable additives are for example flavors, colors, antioxidants, vitamins, sweeteners, such as glucose or aspartam. These oral application systems can also be prepared as retard system by using, for example, Eudragit® or as gastrointestinal therapeutic system or oral osmotic system. The spatial liberation of the drug within the stomach or gut could be varied by the usage of coated tablets with different coats having different solubility.

Preference is given to pharmaceuticals which bring about an increase in the quantity and/or function, particularly the activity, of ACT and AAT polypeptides or functional variants thereof, especially preferably of AAT polypeptides or functional variants thereof. The polypeptides can be prepared synthetically or recombinantly or can be isolated from tissues or cells, with particular preference being given to preparation using an above-described expression system, in particular using E. coli or yeast cells and purification from tissue, particularly serum. The recombinant proteins which have been prepared in this way can also be present as fusion proteins, e.g. so as to facilitate purification or detection.

Another preferred embodiment of a pharmaceutical according to the present invention is the administration of an activating antibody or fragments thereof which increases the activity of an ACT polypeptide in combination with an activating antibody or fragments thereof which increases the activity of an AAT polypeptide, according to the present invention. The application of such a catalytic antibody can be performed as described above.

Administration of the therapeutically active compounds ACT and AAT can be performed together or spatially and/or temporally separated. It is, for example possible to apply a formulation containing ACT or a functional variant thereof separately from a formulation containing AAT or a functional variant thereof. Preferably ACT and AAT polypeptides are formulated together. The concentrations necessary to exert a therapeutic effect may be identical, similar or different.

In another preferred embodiment, use is made of cells which contain at least one nucleic acid encoding an ACT polypeptide or functional variants thereof or nucleic acids encoding these, in combination with at least one nucleic acid encoding an AAT polypeptide or functional variants thereof or nucleic acids encoding these for producing a pharmaceutical for treatment and/or prevention of diseases which are selected from diabetes-associated wounds which heal poorly and/or arterial wounds which heal poorly.

Particular preference is given to cells which can be used in accordance with the invention and which contain the nucleic acids in the form of an above-described expression vector or vector which is applicable in gene therapy. In another preferred embodiment a cells expressing fusion proteins useable according to the invention, or antibodies or a fragments thereof useable according to the invention, are used. The cells can then be introduced into the wound directly or, where appropriate, combined with suitable carrier systems and/or additives and/or auxiliary substances and then introduced into the wound. Suitable carrier systems have been disclosed, for example, in U.S. Pat. No. 5,980,888, WO 92/06179, EP 0242 270 or WO 90/02796. Preferred cells are autologous or allogenic cells, especially preferred are skin cells, in particular keratinocytes, endothelial cells and fibroblasts. In a preferred embodiment, cells expressing both ACT and AAT polypeptides can be used according to the invention. In another preferred embodiment cells expressing ACT polypeptides or functional variants thereof are combined with cells expressing AAT polypeptides or functional variants thereof.

Particular preference is given to pharmaceuticals, for the gene therapy treatment, which comprise nucleic acids which can be used in accordance with the invention and which are contained in a vector or a cell, as explained above, or which comprise polypeptides which can be used in accordance with the invention.

Preference is given to the treatment and prevention of diabetic ulcer and/or arterial ulcer or of a poorly healing wound in a diabetic patient or of a poorly healing wound in a patient suffering from arteriosclerotic destruction of the blood vessels. Especially preferred are chronic diabetic or arterial wounds. In a even more preferred embodiment, the wounds are diabetic ulcer or an arterial ulcer. In a most preferred embodiment, the wound is a diabetic ulcer.

The invention will now be further clarified with the aid of the following tables and examples without it being restricted thereto.

### Tables and Sequences

Table 1: Mean values for the changes in the tensile strength of the wounds in normal and diabetic rats which were treated with mACT (spi2-2) by means of gene therapy, relative to the tensile strength of wounds which were treated with a control vector. The E/C value is the quotient of the absolute tensile strength measured in a rat treated with ACT (E) and the absolute tensile strength measured in a rat treated with a control vector (C).

Table 2: Comparison of the relative amount of active ACT protein within human wound exudates between normally healing wounds and those deriving from different poorly healing wounds as indicated which is an example of a reduced activity selectively occurring in diabetic-associated poorly healing wounds.

SEQ ID No. 1, SEQ ID No.: 3, SEQ ID No. 4, SEQ ID No. 6, SEQ ID No. 7, SEQ ID No. 9, SEQ ID No. 10, SEQ ID No. 11, SEQ ID No. 12, SEQ ID No. 13, SEQ ID No. 14, SEQ ID No. 15 show the sequences of polypeptides which can be used in accordance with the invention.

SEQ ID No. 2, SEQ ID No. 5 and SEQ ID No. 8 show the sequences of nucleic acids which can be used in accordance with the invention.

In particular, SEQ ID NO: 1 shows the amino acid sequence of the mouse serine protease inhibitor 2-2 (spi 2-2);

SEQ ID NO: 2 shows the coding sequence of spi 2-2;

SEQ ID NO: 3 shows the amino acid sequence of the most frequently occuring human allele of AAT having a signal peptide sequence;

SEQ ID NO: 4 shows the sequence of the mature AAT-polypeptide lacking part of the signal peptide;

SEQ ID NO: 5 shows the coding sequence for the mature AAT-polypeptide of SEQ ID NO: 3;

SEQ ID NO: 6 shows the amino acid sequence of human ACT-polypeptide having a signal peptide sequence;

SEQ ID NO: 7 shows a mature human ACT-polypeptide sequence without signal peptide;

SEQ ID NO: 8 shows the nucleic acid coding sequence for human ACT, coding for SEQ ID NO: 6;

SEQ ID NO: 9 shows the amino acid sequence of the Lex032 polypeptide;

SEQ ID NO: 10 shows the amino acid sequence of an in vivo predominant, mature ACT-polypeptide;

SEQ ID NO: 11 shows the amino acid sequence of SEQ ID NO: 7 plus an additional N-terminal methionine;

SEQ ID NO: 12 shows the amino acid sequence of SEQ ID NO: 10 plus an additional N-terminal methionine;

SEQ ID NO: 13 shows the amino acid sequence of AAT according to SEQ ID NO: 4 plus an additional N-terminal methionine;

SEQ ID NO: 14 shows the amino acid sequence of the mature AAT-polypeptide; and

SEQ ID NO: 15 shows the AAT amino acid sequence of SEQ ID NO: 14 plus an additional N-terminal methionine.

Reference is now made to the figures, wherein

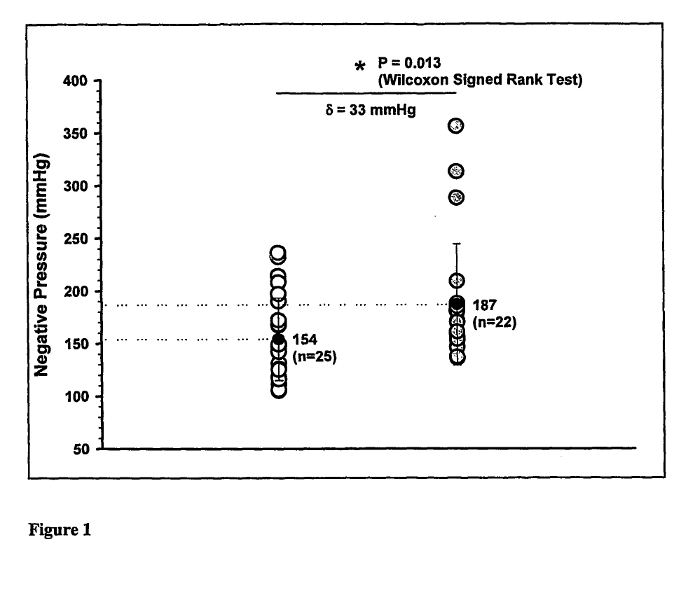

FIG. 1 shows the validation of the human ACT gene in the diabetic mouse model, as described in example 3,

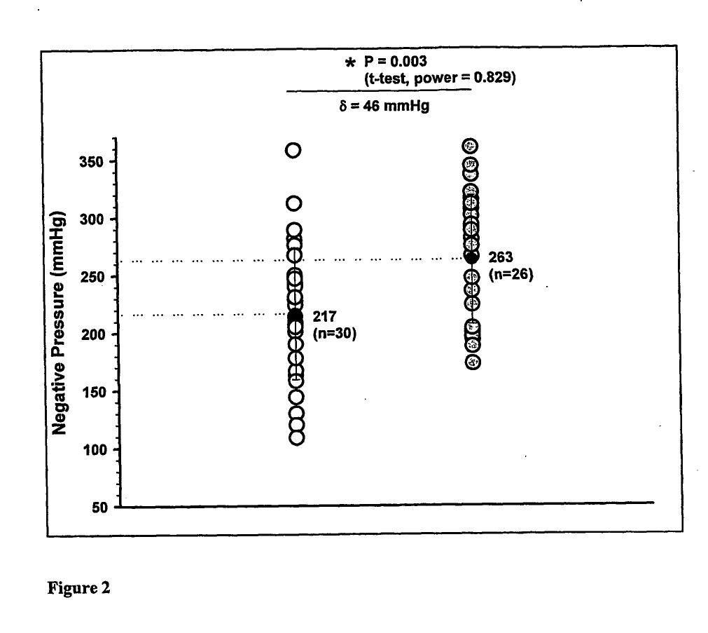

FIG. 2 shows the validation of the human ACT protein in the diabetic mouse model as described in example 4.

In particular, in FIG. 1 before wounding, mice were transfected using the gene gun (400 psi, 1.0 μm Ø of gold particles, 0.5 μg DNA/shoot, 2 shoots/wound) with either the control or the experimental expression plasmid containing the human ACT gene. Immediately following in vivo transfection, one full-thickness 10 mm incisional wound was set through the center of the target tissue site vertical to the spine. On day 5 after wounding tensile strength of incisions was measured by the BTC-2000 system in diabetic mice. The data are expressed as the maximum of negative pressure (mmHg) required to induce breaking of the wound and as mean negative pressure (±SD). Statistical analysis was performed by Wilcoxon Signed Rank Test. Open circles: control wounds, gray circles: ACT treated wounds, black circles: mean values of data

In FIG. 2, 1 dose (0.4 mg/ml) of the human ACT protein in an aqueous buffered solution was tested in impaired healing of incision wounds in diabetic mice. Single wounds were treated with 75 μl protein solution, 30 μl was injected s.c. parallel to each of the two wound margins (1 injection site per wound margin) and 15 μl was applied topically onto the wound. Control animals received only buffer without protein. Protein or buffer was applied two times at day 1 and 2 after wound setting. On day 5 after wounding tensile strength of wounds was measured by the BTC-2000 system. The data are expressed as the maximum of negative pressure (mmHg) required to induce breaking of the wound and as mean negative pressure (±SD). Statistical analysis was performed by Students-t test. Open circles: control wounds, gray circles: wounds treated with human ACT protein, black circles: mean values of data.

### EXAMPLES

### Example 1

### Improving the Wound Healing in Diabetic Rats by Administering the Murine ACT Homologue in vivo

Wound healing was investigated after administering the murine ACT gene, according to SEQ ID No. 2, to diabetic male Sprague Dawley rats. In order to quantify the wound healing, the tensile strength of the wounds was investigated, with a higher tensile strength reflecting an improvement in wound healing.

The diabetic rat animal model is an established model system for investigating diabetes-associated wounds which heal poorly (Davidson, Arch. Dermatol. Res. 290: S1-S11). Since diabetes is accompanied by microangiopathy, this animal model is also suitable for investigating arterially determined disturbances in wound healing.

A suitable expression vector, pMHint, which was prepared on the basis of the vector pMH (S. Hoffman-La Roche), was first of all constructed by inserting intron II of the rat insulin gene into the HindIII cleavage site between the CMV promoter and the multiple cloning site. The mACT cDNA was then cloned into pMHint using the multiple cloning site. For this, the coding region of the mACT cDNA was amplified by PCR (mACT primer 1: 5′GAGGTACCATGGCTTTCATTGCAG 3′ and mACT primer 2: 5′GAATCACGTGACCACCTCCTTTGGGGTTGG CTATC 3′), then cut with KpnI and PmlI and ligated to the expression vector pMHint which had been cut with KpnI and PmlI, thereby giving rise to the expression plasmid pMHintACT. pMHint which contained a luciferase gene (pMHIntLuc) was used as the control vector.

In order to induce the diabetes, 4 rats having a bodyweight of 250-300 g were injected i.p. with a freshly prepared aqueous solution of streptozotocin (Sigma) (50 mg/kg of bodyweight). The blood sugar of the animals was checked 7-9 days after induction, with a blood sugar level value of more than 200 mg/dL confirming the diabetic state.

The 4 diabetic rats and the 4 nondiabetic control animals were subsequently anaesthetized with a mixture consisting of 2% O₂ (2 l/min) and 1.25% isofluran. The back was depilated and 4 sites were marked on the back of each animal for subsequent wounding. In each case 0.5 μg of plasmid DNA immobilized on gold particles (BioRad) was shot into each site at 500 psi using a Helios gene gun (BioRad), with in each case 2 sites being bombarded with the ACT expression vector pMHIntACT and 2 sites being bombarded with the control vector pMHIntLuc and with in each case one bombardment with pMHIntACT being effected anteriorly and one bombardment being effected posteriorly. Incision wounds of 1 cm in length were then made through the bombarded sites and the wounds were closed with wound clips. The wound biopsies were taken after 10 days and the tensile strength of the wounds was determined using an Instron tensiometer in accordance with the manufacturer's instructions and standardized to the cross sectional area of the wounds. Subsequently, the quotient (E/C value) was calculated from the absolute value of the tensile strength of a wound which had been bombarded with pMHInt ACT and the absolute value of the tensile strength of a wound in the same animal which had been bombarded with the control vector pMHIntLuc. The mean of the E/C values was determined and the changes in tensile strength in dependence on mACT thereby ascertained. The means are given in Table 1.

It turned out that the tensile strength of the wounds treated with pMHIntACT was only clearly increased in the diabetic animals whereas the administration of this plasmid had no significant effect in the control animals. This highlights that the deregulation of expression of ACT in poorly healing, diabetes-associated and/or arterial poorly healing wounds can be specifically compensated for by administration of ACT and leads to a pronounced improvement of the wound healing. On the other hand, this treatment was not suitable for improving wound healing in the control animals, in which it was not possible to achieve any significant improvement in wound healing by administering mACT.

### Example 2

### Determination of ACT Activity in Human Wound Exudates of Normally Healing Wounds, Poorly Healing Venous Ulcers and Wounds of Diabetic Patients

Activity of ACT polypeptides was measured in wound exudates from patients with different poorly healing wounds and compared to the activity found in wound exudates derived from acute normally healing wounds after surgery.

The measurement of activity of ACT polypeptides was performed by an activity assay according to Heidtmann et al., 1990, Clinical Chemistry 36: 2077-2081. The principle of this assay comprises the coating of a 96 well-microtiter plates with cathepsin G. Cathepsin G can bind active ACT molecules from wound exudates. After complexing active ACT polypeptides with cathepsin G, ACT polypeptides are detected by a primary rabbit anti-human antibody against ACT polypeptides, followed by a secondary goat anti-rabbit antibody which is coupled to alkaline phosphatase. The amount of bound, active ACT polypeptide is then detected by the application of a chromogenic substrate for alkaline phosphatase by monitoring the increase in absorbance 490 nm in a Versamax Plate reader (Molecular Devices).

To start this experiment, wound exudates from one patient suffering from a venous ulcer and wound exudates from wounds from 2 diabetic patients were collected for a period of 24 h and 48 h, respectively, as examples for poorly healing wounds. As a control, wound exudates from one acute normally healing patient were also collected for 24 h and 48 h, respectively. Those wounds typically arise in the course of surgeries aiming at a mamma reduction. The wound exudates from poorly healing wounds were collected by vacuum therapy: a vacuum extractor was applied to the corresponding wound together with a sponge which equally distributes the pressure on the wound. The aspirated fluid was collected by a bottle. In contrast, the wound exudates which derive from wounds after the acute surgical control wounds were collected by a drainage system which is located within the subcutaneous tissue of the wound and which usually applied to post-surgical wounds to prevent infections (Redon's suction drainage). Subsequently, collected wound exudates were centrifuged in 10 ml Falcon tubes at 1500 rpm at 4° C. in a Heraeus Multifuge 3 S-R for 10 min. to remove contaminating cells. The centrifugation was once repeated with the supernatant at 10000 rpm at 4° C. for 15 min to complete purification. Purified wound exudates from different patients were than diluted 1:5000, 1:10000, and 1:20000 in PBS-Tween to perform the activity assay. Therefore, 96 well-microtiter plates were first coated with 200 μl bovine serum albumin solution (10 g/l BSA in 50 mM NaHCO3, pH 9.6), covered with plate sealers (Qiagen) and incubated for 30 min. at 37° C. Afterwards, BSA solution was aspirated and microtiter plates were washed 4 times with 250 μl PBS for 2 min. The washing solution was then replaced by 100 μl coupling solution which was prepared by 1000 fold dilution of a stock solution (3.3 g/l cathepsin G (Calbiochem) in 50 mM sodium acetate, pH 5.5 and 0.5 M NaCl). Again the incubation period was 30 min. at 37° C. and wells were washed 4 times with 250 μl PBS-Tween for 2 min. To control the coupling of cathepsin G in general, 100 μl of a cathepsin G substrate solution (200 μg/l succinyl-alanyl-alanyl-proplyl-valyl-p-nitroanilide in 0.1 M 4-(2-hydroxyethyl)-1-piperazineethanesulfonate buffer, pH 7.5, 1 M NaCl, 12% DMSO) were added to the microtiter plate. After an incubation period at 37° C. for-100 min., the increase in absorbance at 414 nm was measured in a Versamex plate reader. To control the quantitive amount of cathepsin G coupled to the microtiter plate after the 30-min. incubation period the activity of cathepsin G in the coupling solution was compared with and without incubation in the microtiter plate. Therefore, 100 μl substrate solution (5 g/l succinyl-alanyl-alanyl-proplyl-valyl-p-nitroanilide in DMSO) were added to 900 μl of the coupling solution before and after contacting the microtiter plate. The increase in absorbance was measured for both solutions at 414 nm at 25° C. for 1 min. Results were within the linearity of the assay.

Cathepsin G pre-bound microtiter plates were incubated with 100 μl diluted wound exudates from the acute normally healing wounds, wound exudates from the venous ulcer, wound exudates from wounds of the two diabetic patients, and as a control without addition of any wound exudates. Samples from all patients and all concentrations were applied to the microtiter plate in duplicate. The incubation period again took place at 37° C. with sealed microtiter plates for 30 min., followed by repeated washing with PBS-Tween. After the incubation period, microtiter plates were incubated with 100 μl rabbit anti-human ACT antibody (Dako; 1:1000 in PBS-Tween, 2.5% dry milk) at 37° C. for 30 min. and washed 4 times with PBS-Tween. Incubation of the primary antibody was followed by incubation with a 100 μl of a secondary goat anti-rabbit antibody which was coupled to alkaline phosphatase (Promega, 1:5000 in PBS-Tween, 2.5% dry milk) at 37° C. for 30 min., followed by the washing procedure as described above. As a negative control of the antibody, wells on the microtiter plate were prepared which contain either only wound exudates, only primary antibody, only secondary antibody, only wound exudates with primary antibody, only wound exudates with secondary antibody, or only PBS-Tween, respectively. Alkaline phosphatase was detected by incubation at room temperature with 100 μl/well of OPD (Dako; 6.6 mg/l containing 420 μl/l 30% H₂O₂). The reaction was stopped by adding 100 μl of 0.5M H₂SO₄ per well and absorbance was measured at 490 nm with the above-described ELSIA reader. In parallel, a calibration series was determined by diluting commercially available ACT (Calbiochem; 1 mg/ml in 20 mM Tris, pH 7.4 and 150 mM NaCl) stepwise. A standard curve was determined by plotting the dilution series (1:5000, 1:8000, 1:10000, 1:12000, 1:20000; 1:30000, 1:40000, 1:50000, 1:80000, 1:100000, and 1:120000) against the absorbance measured at 490 nm. Subsequently, the amount of active ACT polypeptides in the different wound exudates was determined by means of the standard curve. The numerical values were than standardized by setting the mean value of the amount of active ACT polypeptides measured in the wound exudates collected for a period of 24 h from acute normally healing wounds equal to 1. The relative changes of the mean value of active ACT polypeptides measured in the wound exudates from the different patients after different collection periods are depicted in Table 2.

The results clearly show a 3.3- and 2.3-fold reduced amount of active ACT polypeptides in poorly healing wounds of diabetic patients compared to acute normally healing wounds. Moreover, the results demonstrate that the reduction of active ACT is essentially selective to poorly-healing, diabetes-associated wounds as the amount of active ACT in wound exudates of poorly healing venous ulcers is within the increasing range of acute normally healing wounds. This reduction of active ACT in poorly-healing, diabetes associates wounds is attributed to the presence of high amounts of elastase in these wounds.

Together, the experiments 1 and 2 demonstrate that ACT is effective for treating diabetes-associated and arterial badly healing wounds, but is sensitive to inactivation and thus, ACT in combination with AAT surprisingly represents an effective and stable therapeutic for the treatment and/or prevention of diabetes-associated and arterial badly healing wounds.

For prevention and/or treatment, the amount and/or the activity of ACT and AAT, especially of ACT has to be increased in the region of the wound. Indications which are preferably to be treated are diabetic ulcer and arterial ulcer, in particular diabetic ulcer. The administration of a pharmaceutical comprising ACT in combination with AAT is preferably effected topically, preferably by means of gene therapy. The administration can furthermore preferably be effected by means of the topical application of an ACT polypeptide in combination with an AAT polypeptide according to the invention, since the site of action of ACT and AAT polypeptide is extracellular and it is consequently not necessary for the proteins to penetrate into cells. Preferably, the polypeptides are isolated from tissue, particularly serum. E.g. U.S. Pat. No. 4,697,003, U.S. Pat. No. 4,439,358, U.S. Pat. No. 4,379,087, WO 02/48176A1, WO 01/38354A1 and U.S. Pat. No. 4,656,254 describe a method for isolating AAT from serum. In a further preferred embodiment, the polypeptides are produced recombinantly, preferably in bacteria, yeast or mammalian cells. ACT and AAT polypeptides may be glycosylated or unglycosylated.

TABLE 1
Mean E/C values

| Control animals | Diabetic animals |
|---|---|
| 1.142 | 3.397 |

TABLE 2

| Source of human | Time Period of wound | Relative amount of |
|---|---|---|
| wound exudate | exudate collection | active ACT protein |
| Surgical wound after | 24 h | 1 |
| mamma reduction | | |
| Surgical wound after | 48 h | 1.6 |
| mamma reduction | | |
| venous ulcer | 24 h | 1.4 |
| venous ulcer | 48 h | 1.2 |
| chronic wound of | 24 h | 0.3 |
| diabetic patients | | |
| chronic wound of | 48 h | 0.7 |
| diabetic patient | | |

### Example 3

### Improving the Wound Healing in Diabetic Mice by Administering the Human ACT Gene

Human α1-Antichymotrypsin (ACT) gene according to SEQ ID NO. 8 was cloned in the in the same expression vector pMHint as described in example 1. The effect human ACT gene on the wound healing was measured by the BTC-2000™ system by determining tensile strength.

10 weeks old female diabetic (C57BL/KsOlaHsd-Lepr db/db) mice were anaesthetized and fine hair of the back was removed with a commercial depilation cream (Pilca). After cleaning the skin with 70% isopropyl alcohol, the site to be wounded (one per mouse in the dorso-cranial area) was transfected using the gene gun (400 psi, 1.0 μm Ø of gold particles, 0.5 μg DNA/shoot, 2 shoots/wound area) with either the control (pMHInt) or the experimental expression plasmid pMHInt containing the human ACT gene. Immediately following in vivo transfection, one full-thickness 10 mm incisional wound was set through the center of the target tissue site vertical to the spine. Wounds were closed with two small strips of a wound plaster (Fixomull stretch, Beiersdorf, Hamburg). At day 5 after wounding the mice were euthanized with 0.1 ml Narcorene. To determine relative breaking strength, incisions were analyzed with an in situ tensiometric device, (BTC-2000™ system, Surgical Research Laboratory, Inc., Nashville, Tenn.).

Wounds, that were not adapted appropriately, were excluded from the calculation of the tensile strength. The results are summarized in FIG. 1. The data are expressed as the maximum of negative pressure (mmHg) required to induce breaking of the wound on day 5 and as mean negative pressure (±SD, standard deviation) for all groups. Statistical analysis was performed by Wilcoxon Signed Rank Test. A P value of less than 0.05 was considered to indicate statistical significance. For the statistical analysis 25 control wounds and 22 wounds treated with human ACT gene could be included. A significant increase in tensile strength was observed in wounds treated with human ACT gene compared to their vector control wounds in diabetic animals at day 5 after wound setting (Negative pressure in mmHg mean±SD: 186.68±57.69 vs 153.96±39.11; P=0.013, Wilcoxon Signed Rank Test, see FIG. 1), proving the beneficial effect of human ACT on healing of diabetes-associated wounds.

### Example 4

### Improving the Wound Healing in Diabetic Mice by Administering Human ACT Protein

One dose of the mature human ACT protein (0.4 mg/ml, α1-Antichymotrypsin from Calbiochem, Order# 178196, corresponding to SEQ ID No. 10) was tested in an aqueous buffered solution in impaired healing of incisional wounds in diabetic mice. The effect human ACT gene on the wound healing was measured by the BTC-2000™ system by determining tensile strength.

10 weeks old female diabetic (C57BL/KsOlaHsd-Lepr db/db) mice were anaesthetized and fine hair of the back was removed with a commercial depilation cream (Pilca). After cleaning the skin with 70% isopropyl alcohol, one full-thickness 10 mm incisional wound was set in the dorso-cranial area vertical to the spine. Wounds were closed with two small strips of a wound plaster (Fixomull stretch, Beiersdorf, Hamburg). At day 5 after wounding the mice were euthanized with 0.1 ml Narcorene. To determine relative breaking strength, 5 day incisions were analyzed with an in situ tensiometric device, (BTC-2000™ system, Surgical Research Laboratory, Inc., Nashville, Tenn.).

The lyophilized ACT protein was reconstituted in an aqueous buffered solution (0.4 mg/ml, composition of the filtered and autoclaved buffer: 50 mM Potassium Phosphate pH 7.2, ionic strength was adjusted to 154 mM with KCl). Single wounds were treated with 75 μl protein solution, 30 μl was injected s.c. parallel to each of the two wound margins (1 injection site per wound margin) and 15 μl was applied topically onto the wound. Control animals received only buffer without protein. Protein or buffer was applied two times at day 1 and 2 after wound setting. On day 5 after wounding tensile strength of wounds was measured by the BTC-2000 system in diabetic mice. Wounds, that were not adapted appropriately, were excluded from the calculation. The results are summarized in FIG. 2. The data are expressed as the maximum of negative pressure (mmHg) required to induce breaking of the wound on day 5 and as mean negative pressure (±SD, standard deviation) for all groups. Statistical analysis was performed by Students-t test. A P value of less than 0.05 was considered to indicate statistical significance. For the statistical analysis 30 control wounds and 26 wounds treated with human ACT could be included. A significant increase in tensile strength was observed in wounds treated with human ACT compared to their control wounds in diabetic animals at day 5 after wound setting (Negative pressure in mmHg mean±SD: 263.00±55.67 vs 216.53±57.11; P=0.003, Students-t test, see FIG. 2), proving the beneficial effect of human ACT on healing of diabetes-associated wounds.

It will be apparent to those skilled in the art that various modifications can be made to the compositions and processes of this invention. Thus, it is intended that the present invention cover such modifications and variations, provided they come within the scope of the appended claims and their equivalents.

1. A method for the treatment and/or prevention of a poorly healing diabetes-associated and/or poorly healing arterial wound, wherein said method comprises administering, to a patient in need of such treatment, an alpha 1-antichymotrypsin (ACT) polypeptide, a functional variant thereof and/or a nucleic acid encoding it, or a cell which is expressing an ACT polypeptide or a nucleic acid encoding it, in combination with an alpha-1-antitrypsin (AAT) polypeptide, a functional variant thereof or a nucleic acid encoding it, or with a cell which is expressing an AAT polypeptide or a nucleic acid encoding it.

2. The method according to claim 1 wherein an ACT polypeptide having a sequence selected from the group consisting of SEQ ID No.: 1, SEQ ID No.: 6, SEQ ID No.: 7, SEQ ID No. 10, SEQ ID No. 11 and SEQ ID No. 12, or a functional variant thereof or a nucleic acid encoding it, is combined with an AAT polypeptide having a sequence selected from the group consisting of SEQ ID No.: 3, SEQ ID No.: 4, SEQ ID No. 13, SEQ ID No. 14 and SEQ ID No. 15, or a functional variant thereof or a nucleic acid encoding it.

3. The method according to claim 1 wherein an ACT polypeptide having a sequence selected from the group consisting of SEQ ID No.: 7, SEQ ID No. 10, SEQ ID No. 11 and SEQ ID No. 12, is combined with an AAT polypeptide having a sequence selected from the group consisting of SEQ ID No.: 4, SEQ ID No. 13, SEQ ID No. 14 and SEQ ID No. 15.

4. The method according to claim 1 wherein a polypeptide having a sequence according to SEQ ID No.: 9 is used.

5. The method according to claim 1, wherein the wound is a diabetic ulcer or an arterial ulcer.

6. The method according to claim 5, wherein the wound is a diabetic ulcer.

7. The method according to claim 1, wherein the polypeptides are unglycosylated.

8. The method according to claim 1, wherein the polypeptides are isolated from tissue.

9. The method according to claim 1, wherein the ACT and AAT polypeptides are administered separately.

10. The method according to claim 1, wherein the nucleic acid is employed in the form of an expression vector.

11. The method according to claim 10, wherein the expression vector is a vector applicable in gene therapy.

12. The method according to claim 1, wherein the cells are autologous or allogenic cells.

13. The method according to claim 12, wherein the cells are skin cells.

14. A pharmaceutical composition for treatment and/or prevention of a disease selected from poorly healing diabetes-associated wounds and poorly healing arterial wounds, wherein said composition comprises an ACT polypeptide, or a nucleic acid coding for the ACT polypeptide, or a cell expressing the ACT polypeptide or a nucleic acid coding for the ACT polypeptide, combined with an AAT polypeptide, or a nucleic acid coding for the AAT polypeptide, or a cell expressing the AAT polypeptide or a nucleic acid coding for the AAT polypeptide, and wherein said composition further comprises a pharmaceutical carrier.

15. The method according to claim 13, wherein the cells are selected from the group consisting of keratinocytes, fibroblasts and endothelial cells.
